(19) United States
(12) Patent Application Publication
(10) Pub. No.: US 2024/0183780 A1
(43) Pub. Date: Jun. 6, 2024

(54) DUAL-COLOR FLUORESCENCE CROSS-CORRELATION SPECTROSCOPY

(71) Applicant: Regeneron Pharmaceuticals, Inc., Tarrytown, NY (US)

(72) Inventors: Jennifer Nguyen, Pleasantville, NY (US); Zachary Oberholtzer, Stamford, CT (US); Dylan Howie, Ossining, NY (US)

(21) Appl. No.: 18/512,259

(22) Filed: Nov. 17, 2023

Related U.S. Application Data
(60) Provisional application No. 63/429,206, filed on Dec. 1, 2022.

(51) Int. Cl. G01N 21/64; G01N 33/68

(52) U.S. Cl. CPC G01N 21/6428; G01N 21/6458; G01N 33/6845; G01N 2021/6439

(57) ABSTRACT

The present invention generally pertains to methods of determining protein-ligand stoichiometries. In particular, the present invention pertains to the use of fluorescence correlation spectroscopy (FCS) to quantify the cross-correlation and auto-correlation times of proteins and their ligands, which can then be used to determine their protein-ligand hydrodynamic radius.

### CROSS-REFERENCE TO RELATED APPLICATIONS

This application claims the benefit of U.S. Provisional Application No. 63/429,206, filed Dec. 1, 2022, which is incorporated by reference herein in its entirety.

### FIELD

This application relates to methods for detection and quantification of hydrodynamic radii. This application also relates to methods for estimating ligand-drug stoichiometry.

### BACKGROUND

Information about the protein-ligand stoichiometries of therapeutic proteins can inform the safety and efficacy of treatments that use therapeutic proteins. Drug- and dose-switching studies can determine whether switching therapeutic proteins and/or dosages affects the safety and efficacy of treatments that use therapeutic proteins. For example, incorporating an additional non-competing therapeutic monoclonal antibody (mAb) in a treatment already using a therapeutic monoclonal antibody can lead to the formation of large protein-ligand complexes that can affect immunogenicity (e.g., “paper-dolling”). Identification of the off-target proteins and elucidation of the mechanisms of action of therapeutic proteins can also inform the safety and efficacy of treatments that use therapeutic proteins. Therefore, it will be appreciated that a need exists for sensitive in situ methods and systems for detecting and measuring protein-ligand stoichiometries of therapeutic proteins.

### SUMMARY

This disclosure provides a method for determining hydrodynamic radius as well as protein-ligand stoichiometry. In an exemplary embodiment, the method comprises: (a) contacting the sample with at least one unique fluorophore capable of binding to a protein; (b) measuring correlation times of the sample using a confocal microscope; and (c) determining the hydrodynamic radius in the sample based on the correlation times.

In one aspect, said correlation times are determined using fluorescence correlation spectroscopy (FCS).

In one aspect, the method is used to estimate protein-ligand stoichiometry in the sample.

In another aspect, a fit model is used to determine said correlation times.

In yet another aspect, said fit model is chosen from the group consisting of a triplet fit model, translation fit model or a combination thereof.

In one aspect, said protein is an antibody.

In another aspect, said protein is a monoclonal antibody.

In one aspect, two unique fluorophores exhibit non-overlapping emission spectra.

In another aspect, two unique fluorophores comprise Alexa Fluor 488, Alexa Fluor 647 or both.

In one aspect, said correlation times are chosen from the group consisting of cross-correlation times, auto-correlation times or a combination thereof.

In one aspect, said sample is serum.

In another aspect, said sample comprises a biological system.

In another exemplary embodiment, the method for determining protein-ligand stoichiometry or hydrodynamic radius comprises: (a) contacting the sample with at least one unique fluorophore capable of binding to a protein; (b) measuring cross-correlation and/or auto-correlation times of the fluorophores within the sample using a confocal microscope capable of FCS; and (c) determining the hydrodynamic radius in the sample based on the correlation times.

In one aspect, said cross-correlation and/or auto-correlation times are determined using fluorescence correlation spectroscopy (FCS).

In one aspect, the method is used to estimate protein-ligand stoichiometry in the sample.

In another aspect, a fit model is used to determine said cross-correlation and/or auto-correlation times.

In yet another aspect, said fit model is chosen from the group consisting of a triplet fit model, translation fit model or a combination thereof.

In one aspect, said protein is an antibody.

In another aspect, said protein is a monoclonal antibody.

In one aspect, two unique fluorophores exhibit non-overlapping emission spectra.

In another aspect, two unique fluorophores comprise Alexa Fluor 488, Alexa Fluor 647 or both.

In one aspect, said sample is serum.

In another aspect, said sample comprises a biological system.

In another exemplary embodiment, the method for determining protein-ligand stoichiometry or hydrodynamic radius comprises: (a) contacting the sample with at least one unique fluorophore capable of binding to a protein; (b) measuring cross-correlation and/or auto-correlation times of the fluorophores within the sample using a confocal microscope capable of FCS; and (c) estimating the protein-ligand stoichiometry in the sample based on the correlation times.

In one aspect, a fit model is used to determine said cross-correlation and/or auto-correlation times.

In another aspect, said fit model is chosen from the group consisting of a triplet fit model, translation fit model or a combination thereof.

In one aspect, said protein is an antibody.

In another aspect, said protein is a monoclonal antibody.

In one aspect, two unique fluorophores exhibit non-overlapping emission spectra.

In another aspect, two unique fluorophores comprise Alexa Fluor 488, Alexa Fluor 647 or both.

In one aspect, said sample is serum.

In another aspect, said sample comprises a biological system.

In another exemplary embodiment, the method for determining protein-ligand stoichiometry or hydrodynamic radius comprises: (a) labeling a protein with a first fluorophore; (b) labeling a ligand of the protein with a second fluorophore; (c) combining the labeled protein and the labeled ligand in said sample; (d) measuring correlation times of the sample using a confocal microscope capable of FCS; and (e) determining the hydrodynamic radius in the sample based on the correlation times.

In one aspect, said correlation times are determined using fluorescence correlation spectroscopy (FCS).

In one aspect, the method is used to estimate protein-ligand stoichiometry in the sample.

In another aspect, a fit model is used to determine said correlation times.

In yet another aspect, said fit model is chosen from the group consisting of a triplet fit model, translation fit model or a combination thereof.

In one aspect, said protein is an antibody.

In another aspect, said protein is a monoclonal antibody.

In one aspect, said first and second fluorophores exhibit non-overlapping emission spectra.

In another aspect, said first and second fluorophores comprise Alexa Fluor 488, Alexa Fluor 647 or both.

In one aspect, said correlation times are chosen from the group consisting of cross-correlation times, auto-correlation times or a combination thereof.

In one aspect, said sample is serum.

In another aspect, said sample comprises a biological system.

In another exemplary embodiment, the method for determining protein-ligand stoichiometry or hydrodynamic radius comprises: (a) labeling a protein with a first fluorophore; (b) labeling a ligand of the protein with a second fluorophore; (c) combining the labeled protein and the labeled ligand in the sample; (d) measuring cross-correlation and/or auto-correlation times of the sample using a confocal microscope capable of FCS; and (e) estimating the protein-ligand stoichiometry in the sample based on the correlation times.

In one aspect, said cross-correlation and auto-correlation times are determined using fluorescence correlation spectroscopy (FCS).

In another aspect, a fit model is used to determine said cross-correlation and/or auto-correlation times.

In yet another aspect, said fit model is chosen from the group consisting of a triplet fit model, translation fit model or a combination thereof.

In one aspect, said protein is an antibody.

In another aspect, said protein is a monoclonal antibody.

In one aspect, said first and second fluorophores exhibit non-overlapping emission spectra.

In another aspect, said first and second fluorophores comprise Alexa Fluor 488, Alexa Fluor 647 or both.

In one aspect, said sample is serum.

In another aspect, said sample comprises a biological system.

In another exemplary embodiment, the method for determining protein-ligand stoichiometry or hydrodynamic radius comprises: (a) labeling a protein with a first fluorophore; (b) labeling a secondary labeled reporter with a second fluorophore; (c) combining the labeled protein and the secondary labeled reporter in said sample; (d) measuring cross-correlation and/or auto-correlation times of the sample using a confocal microscope capable of FCS; and (e) determining the hydrodynamic radius in the sample based on the correlation times.

In one aspect, a fit model is used to determine said cross-correlation and/or auto-correlation times.

In one aspect, the method is used to estimate protein-ligand stoichiometry in the sample.

In another aspect, said fit model is chosen from the group consisting of a triplet fit model, translation fit model or a combination thereof.

In one aspect, said protein is an antibody.

In another aspect, said protein is a monoclonal antibody.

In one aspect, said first and second fluorophores exhibit non-overlapping emission spectra.

In another aspect, said first and second fluorophores comprise Alexa Fluor 488, Alexa Fluor 647 or both.

In one aspect, said sample is serum.

In another aspect, said sample comprises a biological system.

### BRIEF DESCRIPTION OF THE DRAWINGS

FIG. 1 shows the path light travels from a laser to a detector in a fluorescence correlation spectroscopy (FCS) setup, according to an exemplary embodiment.

FIG. 2 shows the components of a fluorescence correlation spectroscopy setup, according to an exemplary embodiment.

FIG. 3 shows an example of the fluorescent signal (middle pane) and auto-correlation curve (bottom pane) of a fluorescent particle within a focus volume (top pane) using a fluorescence correlation spectroscopy setup, according to an exemplary embodiment.

FIG. 4 shows an example of the correlation of a signal with a delayed copy of itself as a function of delay (e.g., “auto-correlation”), according to an exemplary embodiment.

FIG. 5 shows examples of comparisons of two data series from different spectra for which the degree of similarity as a function of displacement of one relative to another can be quantified (e.g., “cross-correlation), according to an exemplary embodiment.

FIG. 6 shows cross-correlation and auto-correlation curves, according to an exemplary embodiment. On the left, the two source signals are correlated in time and differ in magnitude. On the right, the two source signals are perfectly anti-correlated in time and differ in magnitude.

FIG. 7 shows that the use of two non-competing therapeutic monoclonal antibodies can lead to paper dolling, according to an exemplary embodiment.

FIG. 8 shows Alexa Fluor 488 (top panel), Alexa Fluor 647 (middle panel) and the excitation and emission spectra of Alexa Fluor 488 and Alexa Fluor 647 (bottom panel), according to an exemplary embodiment.

FIG. 9 shows an IgG4 antibody (top panel) composed of two heavy chains (teal) and light chains (light green) and a dimeric ligand of IgG4, nerve growth factor (bottom panel), according to an exemplary embodiment.

FIG. 10 shows the fluorescence correlation spectroscopy auto-correlation curves of free dye, dye-conjugated-mAb1 and mAb1 complexes produced using 1:5 dye-conjugated-mAb1:target, according to an exemplary embodiment.

FIG. 11 shows the fluorescence correlation spectroscopy auto-correlation times of free dye, dye-conjugated-mAb1 and mAb1 complexes produced using 1:5 dye-conjugated-mAb1:target, according to an exemplary embodiment.

FIG. 12 shows the fluorescence correlation spectroscopy auto-correlation curves of free dye, dye-conjugated-mAb1, a dye-conjugated-mAb1 mixture and mAb1 complexes produced using 1:5 dye-conjugated-mAb1:target, 0.5:0.5:5 mAb1-A488:mAb1-A647:target or 2.5:2.5:1 mAb1-A488:mAb1-A647:target, according to an exemplary embodiment.

FIG. 13 shows the fluorescence correlation spectroscopy cross-correlation curves of a dye-conjugated-mAb1 mixture and mAb1 complexes produced using 0.5:0.5:5 mAb1-A488:mAb1-A647:target or 2.5:2.5:1 mAb1-A488:mAb1-A647:target, according to an exemplary embodiment.

FIG. 14 shows the fluorescence correlation spectroscopy auto-correlation and cross-correlation curves of dye-conjugated-mAb1 using 0.5:0.5:5 mAb1-A488:mAb1-A647:target or 2.5:2.5:1 mAb1-A488:mAb1-A647:target, according to an exemplary embodiment.

FIG. 15 shows the fluorescence correlation spectroscopy auto-correlation times of free dye, dye-conjugated-mAb1 and mAb1 complexes produced using 1:5 dye-conjugated-mAb1:target, 0.5:0.5:5 mAb1-A488:mAb1-A647:target or 2.5:2.5:1 mAb1-A488:mAb1-A647:target, according to an exemplary embodiment.

FIG. 16 shows FCS/FCCS results for mAb1:NGF samples, according to an exemplary embodiment.

### DETAILED DESCRIPTION

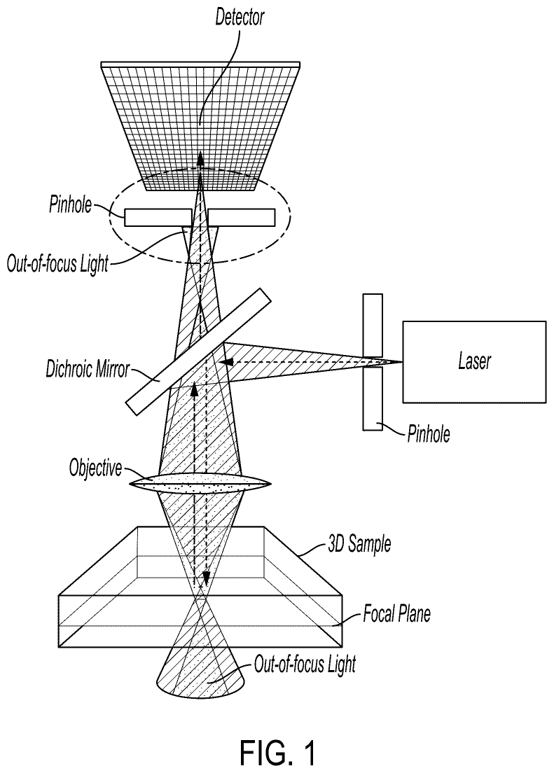

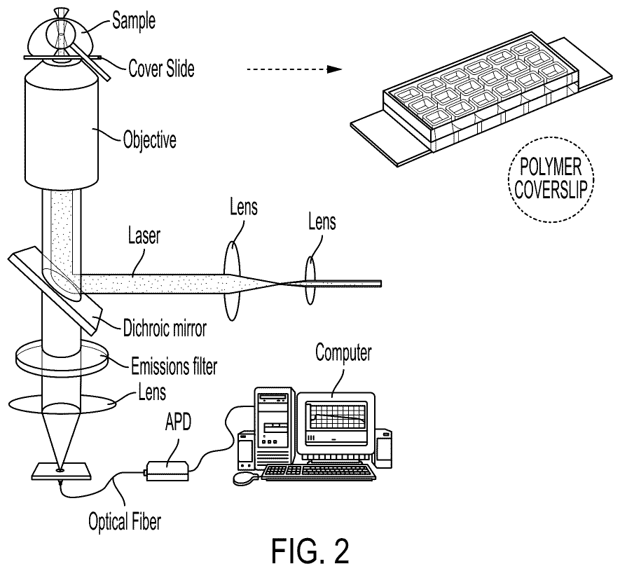

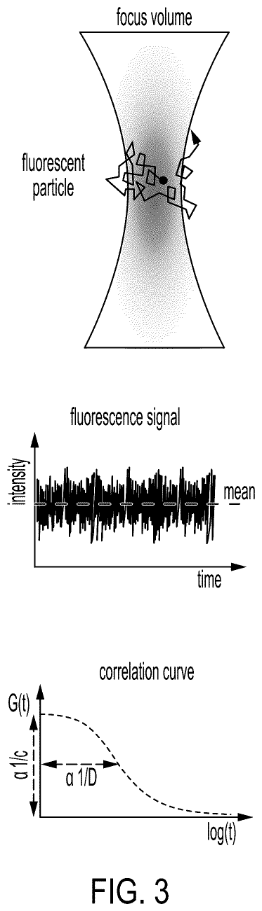

In an exemplary embodiment, fluorescence correlation spectroscopy (FCS) can be used to detect and determine hydrodynamic radius. FCS is analogous to dynamic light scattering spectroscopy (DLS). FCS and DLS can measure the size distribution of particles based on intensity fluctuations of scattered light (e.g., DLS) or fluorescence (e.g., FCS). FCS uses the physical phenomena of fluorescence, whereas DLS uses the physical phenomena of light scattering. In exemplary embodiments, a laser can shine through a dichroic mirror that filters for a precise wavelength of light and reflected on a sample to excite fluorophores of interest, and the emitted light can be reflected through a pinhole that can filter wavelengths of light from outside the sample volume to a detector. In exemplary embodiments, the detector can convert captured light into an electrical signal and transmit the electrical signal to a computer via a cable. In one aspect, the cable is an optical fiber. In yet another aspect, the computer can record intensity fluctuations and apply an auto-correlation Fourier Transform. FIG. 1 and FIG. 2 show exemplary embodiments, in which a laser shines through a polarizer, reflects onto the sample and is converted into an electric signal by a photomultiplier in fluorescence correlation spectroscopy. FIG. 3 shows how the fluorescence signal and correlation curve of a fluorescent particle may generally appear in an exemplary embodiment.

In exemplary embodiments, diffusion coefficients are measured with a confocal microscope capable of FCS. The diffusion coefficients can be used to calculate a hydrodynamic radius using the Stoke-Einstein equation. The protein-ligand stoichiometry can then be estimated from these calculations.

In exemplary embodiments, the benefits of FCS are that only a species of interest may be observed with high sensitivity (e.g., nanomolar concentrations). In another aspect, measurements of protein-ligand stoichiometry can be measured in situ. In yet another aspect, the measurements may comprise one or more cellular systems.

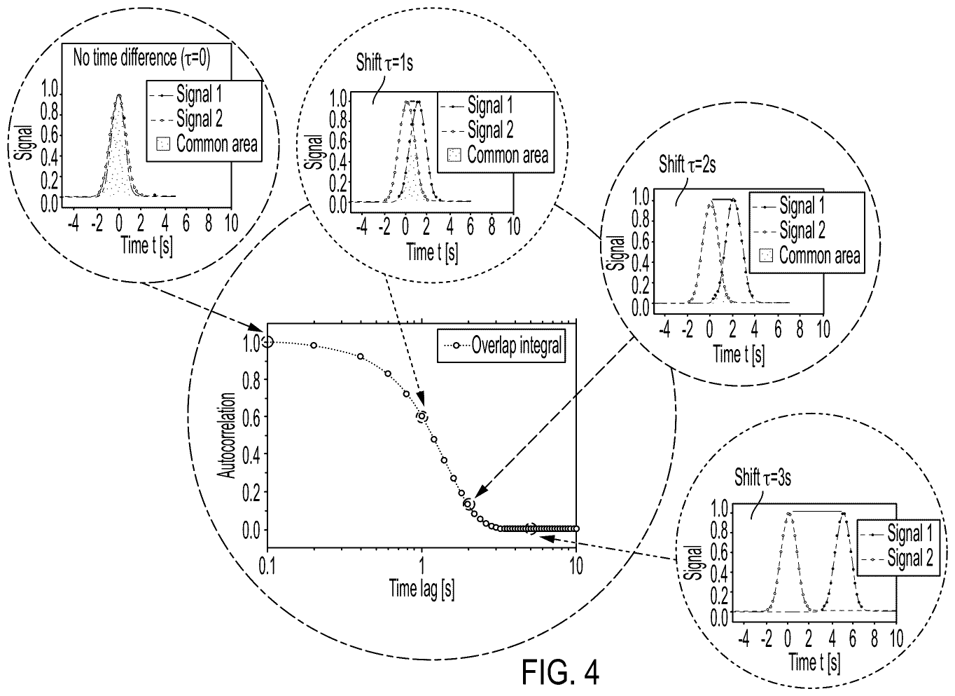

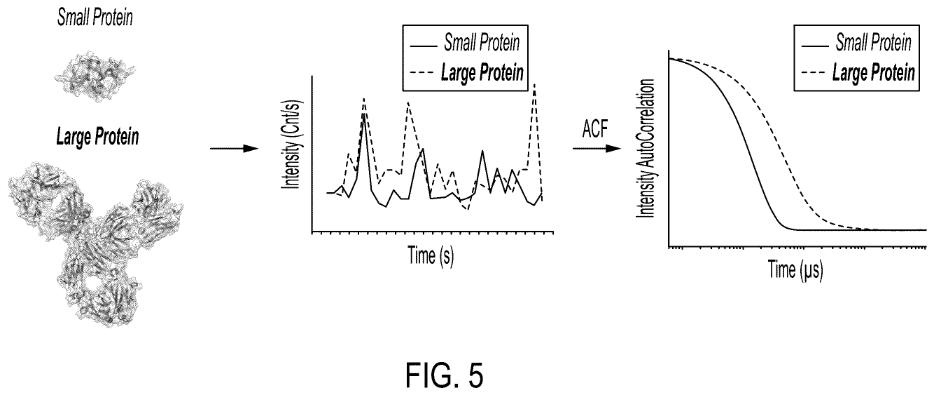

In exemplary embodiments, auto-correlation can be the correlation of a signal with a delayed copy of itself as a function of delay. FIG. 4 shows how an auto-correlation curve can be determined, according to one aspect. Superior auto-correlation data suggests that data remained relatively constant throughout a given delay period. In exemplary embodiments, protein size can affect the diffusion rates of molecules, which can change the shape of the autocorrelation function. In exemplary embodiments, diffusion rates can be inversely proportionate to the sizes of proteins because smaller proteins permeate through liquid more easily and thus diffuse faster. FIG. 5 shows an exemplary embodiment in which a smaller protein has a lower signal intensity and diffuses faster than a larger protein.

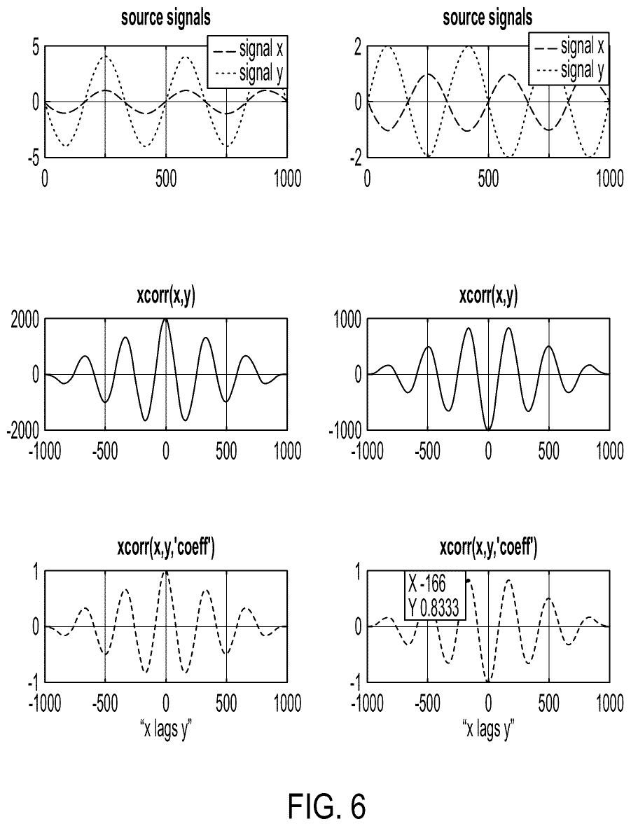

In exemplary embodiments, cross-correlation can compare two or more data series from different spectra and quantify the similarity of one relative to another as a function of the displacement of the one relative to the other. FIG. 6 shows the correlation of data, the source signals on the left are correlated in time, the source signals on the right are perfectly anti-correlated in time.

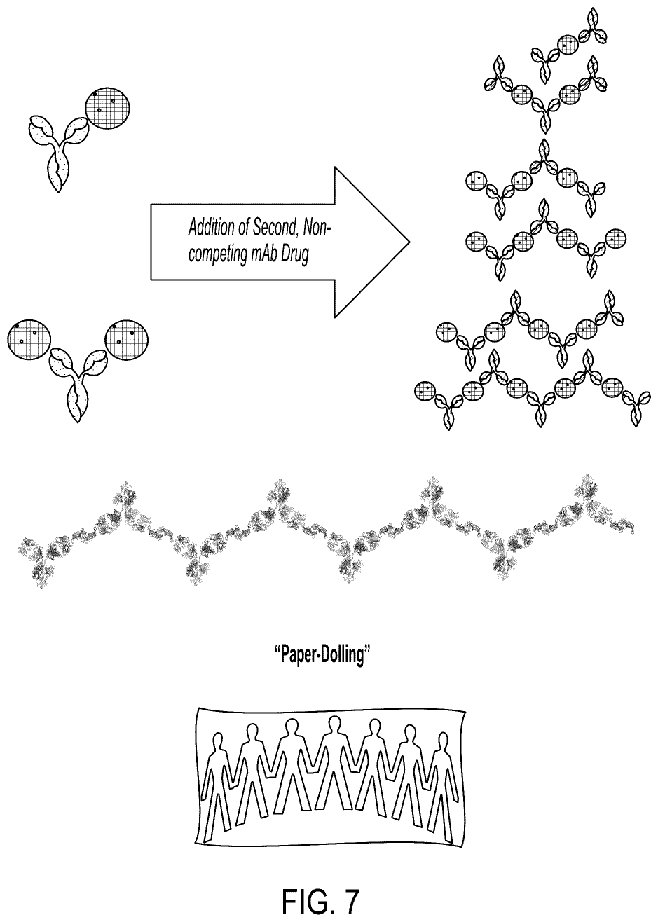

In exemplary embodiments, the binding of therapeutic mAbs to soluble, multimeric targets can lead to large heterogeneous complexes, or “paper-dolling.” Paper-dolling is a phenomenon that commonly occurs when there is an excess of ligand, leading to a larger hydrodynamic radius. Paper-dolling is less likely to occur if there is an excess of antibody. Paper-dolling occurs because two antibodies can attach to the same ligand/target on opposite sides and join together in a large chain, wherein additional ligand can result in more antibodies joining, and newly joined antibodies can attach to more ligands, leading to a feedback loop. In exemplary embodiments, systems capable of paper-dolling are ideal to maximize the difference in correlation lag times observed when ligand is added. FIG. 7 shows an exemplary embodiment in which the antibodies and ligand used are capable of paper-dolling.

Unless described otherwise, all technical and scientific terms used herein have the same meaning as commonly understood by one of ordinary skill in the art to which this invention belongs. Although practice or testing can use methods and materials similar or equivalent to those described herein, particular methods and materials are now described.

The terms “a” and “an” should be understood to mean “at least one” and the terms “about” and “approximately” should be understood to permit standard variation as would be understood by those of ordinary skill in the art and where ranges are provided, endpoints are included. As used herein, the terms “include,” “includes,” and “including” are meant to be non-limiting and are understood to mean “comprise,” “comprises,” and “comprising” respectively.

As used herein, the term “composition” refers to a pharmaceutical product formulated together with one or more pharmaceutically acceptable vehicles.

As used herein, the terms “pharmaceutical” and “pharmaceutical product” can include a biologically active component of a drug product. A pharmaceutical and a pharmaceutical product can refer to any substance or combination of substances used in a drug product, intended to furnish pharmacological activity or to otherwise have a direct or indirect effect on the diagnosis, cure, mitigation, treatment, or prevention of disease, or to have a direct or indirect effect in restoring, correcting or modifying physiological functions in animals. Non-limiting examples of a pharmaceutical or a pharmaceutical product can include a drug, a chemical compound, a nucleic acid, a nucleotide, a nucleoside, an oligonucleotide, a toxin, a peptide, a protein, a fusion protein, an antibody, an antibody fragment, a Fab region of an antibody, an scFv, a monoclonal antibody, a bispecific antibody, a multispecific antibody, an antibody-drug conjugate, or a pharmaceutical protein product, or combinations thereof. Non-limiting examples of processes or elements that can be used in a method of preparing a pharmaceutical or a pharmaceutical product can include a fermentation process, recombinant DNA, isolation and recovery from natural resources, chemical synthesis, biosynthesis, polymerase chain reaction, or combinations thereof.

As used herein, the terms “protein” and “pharmaceutical protein product” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. “Synthetic peptides or polypeptides” refers to a non-naturally occurring peptide or polypeptide. Synthetic peptides or polypeptides can be synthesized, for example, using an automated polypeptide synthesizer. Various solid-phase peptide synthesis methods are known to those of skill in the art. A protein may comprise one or multiple polypeptides to form a single functioning biomolecule. A protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect baculovirus system, yeast systems (e.g., Pichia sp.), mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., Production platforms for biotherapeutic glycoproteins. Occurrence, impact, and challenges of non-human sialylation, 28 Biotechnology and Genetic Engineering Reviews 147-176 (2012), the entire teachings of which are herein incorporated). Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins. Non-limiting examples of a protein or a pharmaceutical protein product can include a recombinant protein, an antibody, a bispecific antibody, a multispecific antibody, an antibody fragment, a monoclonal antibody, a fusion protein, an scFv and combinations thereof.

As used herein, the term “recombinant protein” refers to a protein produced as the result of the transcription and translation of a gene carried on a recombinant expression vector that has been introduced into a suitable host cell. In certain exemplary embodiments, the recombinant protein can be an antibody, for example, a chimeric, humanized, or fully human antibody. In certain exemplary embodiments, the recombinant protein can be an antibody of an isotype selected from group consisting of. IgG (e.g., IgG1, IgG2, IgG3, IgG4), IgM, IgA1, IgA2, IgD, or IgE. In certain exemplary embodiments, the antibody molecule is a full-length antibody (e.g., an IgG1 or IgG4 immunoglobulin), or the antibody can be a fragment (e.g., an Fc fragment or a Fab fragment).

The term “antibody,” as used herein, includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CHi, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FRs). Each VH and VL is composed of three complementarity determining regions and four framework regions, arranged from amino-terminus to carboxy-terminus in the following order: FR1, CDR1, FR2, CDR2, FR3, CDR3, and FR4. In different embodiments of the invention, the framework regions of the anti-big-ET-1 antibody (or antigen-binding portion thereof) may be identical to the human germline sequences or may be naturally or artificially modified. An amino acid consensus sequence may be defined based on a side-by-side analysis of two or more complementarity determining regions. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, an F(ab′)2 fragment, an scFv fragment, an Fv fragment, a dsFv diabody, a dAb fragment, an Fd′ fragment, an Fd fragment, and an isolated complementarity determining region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment comprises a sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single-chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” includes an antibody capable of selectively binding two or more epitopes. Bispecific antibodies generally comprise two different heavy chains, with each heavy chain specifically binding a different epitope-either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains, each having three heavy chain complementarity determining regions, followed by a CHi domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bispecific antibodies (bsAbs) can have different formats such as, but not limited to, triomab, knobs-into-holes IgG (KiH IgG), crossMab, orth-Fab IgG, Dual-variable domains Ig (DVD-Ig), two-in-one or dual action Fab (DAF), IgG-single-chain Fv (IgG-scFv), or κλ-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang and Mingju Hao, Bispecific Antibodies and Their Applications, 8 Journal of Hematology & Oncology 130; Dafne Müller and Roland E. Kontermann, Bispecific Antibodies, Handbook of Therapeutic Antibodies 265-310 (2014), the entire teachings of which are herein incorporated).

As used herein, “multispecific antibody” refers to an antibody with binding specificities for at least two different antigens. While such molecules normally will only bind two antigens (e.g., bispecific antibodies/bsAbs), antibodies with additional specificities such as trispecific antibodies and KiH trispecific antibodies can also be addressed by the system and method disclosed herein.

The term “monoclonal antibody” as used herein, is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art, including the use of hybridoma, recombinant, and phage display technologies, or a combination thereof.

The term “secondary labeled reporter” as used herein, can be anything that binds to a complex of interest. Non-limiting examples include, a secondary mAb (that can bind to the human Fc region of a drug-antibody complex), a Fab, another ligand.

In some exemplary embodiments, a protein and a pharmaceutical protein product can be produced from mammalian cells. The mammalian cells can be of human origin or non-human origin, and can include primary epithelial cells (e.g., keratinocytes, cervical epithelial cells, bronchial epithelial cells, tracheal epithelial cells, kidney epithelial cells and retinal epithelial cells), established cell lines and their strains (e.g., 293 embryonic kidney cells, BHK cells, HeLa cervical epithelial cells and PER-C6 retinal cells, MDBK (NBL-1) cells, 911 cells, CRFK cells, MDCK cells, CHO cells, BeWo cells, Chang cells, Detroit 562 cells, HeLa 229 cells, HeLa S3 cells, Hep-2 cells, KB cells, LSI80 cells, LS174T cells, NCI-H-548 cells, RPM12650 cells, SW-13 cells, T24 cells, WI-28 VA13, 2RA cells, WISH cells, BS-C-I cells, LLC-MK2 cells, Clone M-3 cells, 1-10 cells, RAG cells, TCMK-1 cells, Y-1 cells, LLC-PKi cells, PK(15) cells, GHi cells, GH3 cells, L2 cells, LLC-RC 256 cells, MHiCi cells, XC cells, MDOK cells, VSW cells, and TH-I, B1 cells, BSC-1 cells, RAf cells, RK-cells, PK-15 cells or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, Midi cells, CHO cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C3H/IOTI/2 cells, HSDMiC3 cells, KLN205 cells, McCoy cells, Mouse L cells, Strain 2071 (Mouse L) cells, L-M strain (Mouse L) cells, L-MTK′ (Mouse L) cells, NCTC clones 2472 and 2555, SCC-PSA1 cells, Swiss/3T3 cells, Indian muntjac cells, SIRC cells, Cn cells, and Jensen cells, Sp2/0, NS0, NS1 cells or derivatives thereof).

A composition can be used for the treatment, prevention, and/or amelioration of a disease or disorder. Exemplary, non-limiting diseases and disorders that can be treated and/or prevented by the administration of the pharmaceutical formulations of the present invention include, infections; respiratory diseases; pain resulting from any condition associated with neurogenic, neuropathic or nociceptic pain; genetic disorder; congenital disorder; cancer; herpetiformis; chronic idiopathic urticarial; scleroderma, hypertrophic scarring; Whipple's Disease; benign prostate hyperplasia; lung disorders, such as mild, moderate or severe asthma, allergic reactions; Kawasaki disease, sickle cell disease; Churg-Strauss syndrome; Grave's disease; pre-eclampsia; Sjogren's syndrome; autoimmune lymphoproliferative syndrome; autoimmune hemolytic anemia; Barrett's esophagus; autoimmune uveitis; tuberculosis; nephrosis; arthritis, including chronic rheumatoid arthritis; inflammatory bowel diseases, including Crohn's disease and ulcerative colitis; systemic lupus erythematosus; inflammatory diseases; HIV infection; AIDS; LDL apheresis; disorders due to PCSK9-activating mutations (gain of function mutations, “GOF”), disorders due to heterozygous Familial Hypercholesterolemia (heFH); primary hypercholesterolemia; dyslipidemia; cholestatic liver diseases; nephrotic syndrome; hypothyroidism; obesity; atherosclerosis; cardiovascular diseases; neurodegenerative diseases; neonatal Onset Multisystem Inflammatory Disorder (NOM ID/CINCA); Muckle-Wells Syndrome (MWS); Familial Cold Autoinflammatory Syndrome (FCAS); familial Mediterranean fever (FIMF); tumor necrosis factor receptor-associated periodic fever syndrome (TRAPS); systemic onset juvenile idiopathic arthritis (Still's Disease); diabetes mellitus type 1 and type 2; auto-immune diseases; motor neuron disease; eye diseases; sexually transmitted diseases; tuberculosis; disease or condition which is ameliorated, inhibited, or reduced by a VEGF antagonist; disease or condition which is ameliorated, inhibited, or reduced by a PD-1 inhibitor; disease or condition which is ameliorated, inhibited, or reduced by a Interleukin antibody; disease or condition which is ameliorated, inhibited, or reduced by a NGF antibody; disease or condition which is ameliorated, inhibited, or reduced by a PCSK9 antibody; disease or condition which is ameliorated, inhibited, or reduced by a ANGPTL antibody; disease or condition which is ameliorated, inhibited, or reduced by an activin antibody; disease or condition which is ameliorated, inhibited, or reduced by a GDF antibody; disease or condition which is ameliorated, inhibited, or reduced by a Fel d1 antibody; disease or condition which is ameliorated, inhibited, or reduced by a CD antibody; disease or condition which is ameliorated, inhibited, or reduced by a C5 antibody or combinations thereof.

A composition can be administered to a patient. Administration may be via any route acceptable to those skilled in the art. Non-limiting routes of administration include oral, topical, or parenteral. Administration via certain parenteral routes may involve introducing the formulations of the present invention into the body of a patient through a needle or a catheter, propelled by a sterile syringe or some other mechanical device such as a continuous infusion system. A composition may be administered using a syringe, injector, pump, or any other device recognized in the art for parenteral administration. A composition may also be administered as an aerosol for absorption in the lung or nasal cavity. The solutions may also be administered for absorption through the mucus membranes, such as in buccal administration.

A formulation can further comprise excipients including, but not limited to, buffering agents, bulking agents, tonicity modifiers, solubilizing agents, and preservatives. Other additional excipients can also be selected based on function and compatibility with the formulations may be found, for example, in Remington: The Science and Practice of Pharmacy, (2005); U.S. Pharmacopeia: National formulary; Louis Sanford Goodman et al., Goodman and Gilmans The Pharmacological Basis of Therapeutics (2001); Kenneth E. Avis, Herbert A. Lieberman and Leon Lachman, Pharmaceutical Dosage Forms: Parenteral Medications (1992); Praful Agrawala, Pharmaceutical Dosage Forms: Tablets. Volume 1, 79 Journal of Pharmaceutical Sciences 188 (1990); Herbert A. Lieberman, Martin M. Rieger and Gilbert S. Banker, Pharmaceutical Dosage Forms: Disperse Systems (1996); Myra L. Weiner and Lois A. Kotkoskie, Excipient Toxicity and Safety (2000), herein incorporated by reference in their entirety.

It is understood that the present invention is not limited to any of the aforesaid solution(s), composition(s), pharmaceutical(s), pharmaceutical product(s), protein(s), pharmaceutical protein product(s), protein(s), polypeptide(s), synthetic polypeptide(s), recombinant protein(s), antibody(ies), antigen-binding portion(s), antigen-binding fragment(s), antibody fragment(s), bispecific antibody(ies), multispecific antibody(ies), formulation(s), excipient(s) or cell(s) and solution(s), composition(s), pharmaceutical(s), pharmaceutical product(s), protein(s), pharmaceutical protein product(s), protein(s), polypeptide(s), synthetic polypeptide(s), recombinant protein(s), antibody(ies), antigen-binding portion(s), antigen-binding fragment(s), antibody fragment(s), bispecific antibody(ies), multispecific antibody(ies), formulation(s), excipient(s) or cell(s) can be selected by any suitable means.

### EXAMPLES

Fluorophores with Non-Overlapping Emission Spectra

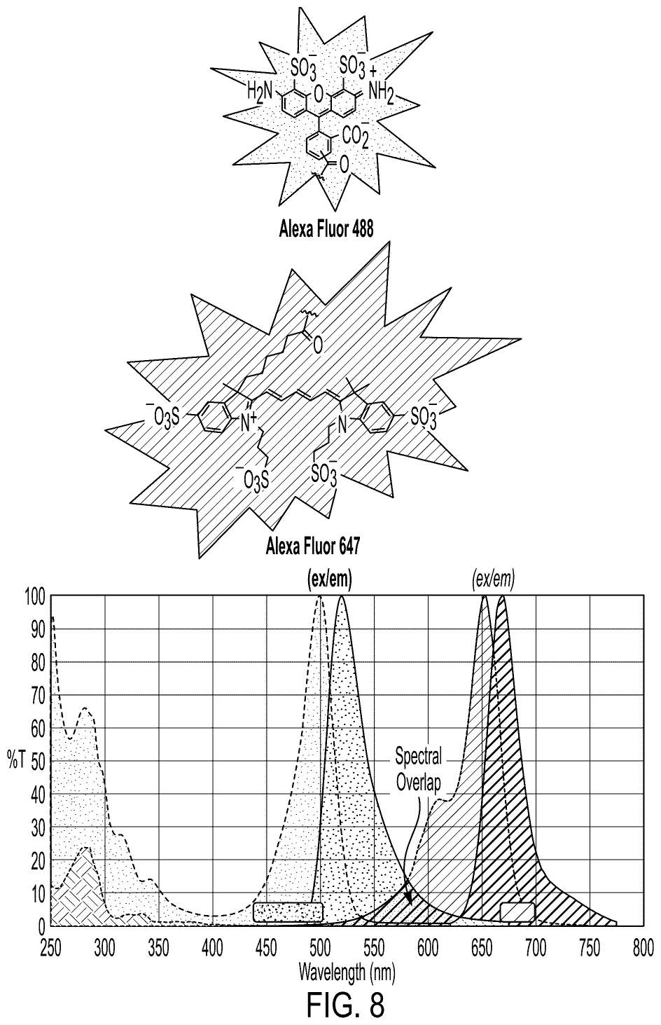

Cross-correlation requires two samples from different emission spectra, which contain different fluorophores. FIG. 8 shows the excitation spectra of Alexa Fluor 488 (A488) (light blue) and Alexa Fluor 647 (A647) (light pink), the emission spectra of Alexa Fluor 488 (blue) and Alexa Fluor 647 (pink) and the overlap of the Alexa Fluor 488 excitation and emission spectra with the Alexa Fluor 647 excitation and emission spectra (green).

### Example 1

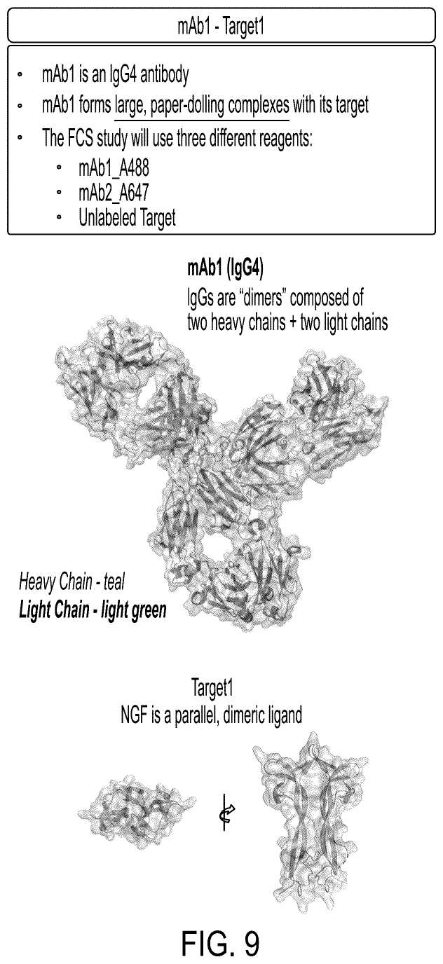

FIG. 9 shows that FCS and three different reagents, IgG4 labeled with Alexa Fluro 488 (mAb1-A488), IgG4 labeled with Alexa Fluor 647 (mAb1-647) and nerve growth factor (NGF) (unlabeled target), were used in one study. IgG4 and nerve growth factor were used for the study because the IgG4 epitopes are on opposite ends of nerve growth factor, which enables paper-dolling. Table 1 shows the study design and sample set used for single-channel FCS. It was predicted that excess mAb1 relative to ligand would lead to larger complexes that resulted in longer correlation times than excess ligand relative to mAb1.

TABLE 1
Study Design/Sample Set for Single-Channel Fluorescence Correlation Spectroscopy

| | Test Article | | | | | | |
|---|---|---|---|---|---|---|---|
| | | | | | | mAb-A488 × | |
| | | | Cross- | mAb-A488 | mAb-A647 | mAb1-647 | mAb-A488 × |
| | 44 Ch. | 647 Ch. | Correlation | Excess | Excess | Excess | mAb1-647 |
| | mAb | mAb | Negative | Ligand | Ligand | Ligand | Excess mAb |
| Reagent | Control | Control | Control | Complexes | Complexes | Complexes | Complexes |
| mAb1-A488 | + | − | + | 1 | − | 0.5 | 2.5 |
| mAb1-A647 | − | + | + | − | 1 | 0.5 | 2.5 |
| Target | − | − | − | 5 | 5 | 5 | 1 |

Hypothesis (Diffusion Time Length)

| | | | | | | | |
|---|---|---|---|---|---|---|---|
| 488 Chanel | + | − | + | ++ | − | ++ | +++ |
| 647 Channel | − | + | + | − | ++ | ++ | +++ |
| Cross- | − | − | − | − | − | +++ | ++++ |
| Correlation | | | | | | | |
| Channel | | | | | | | |

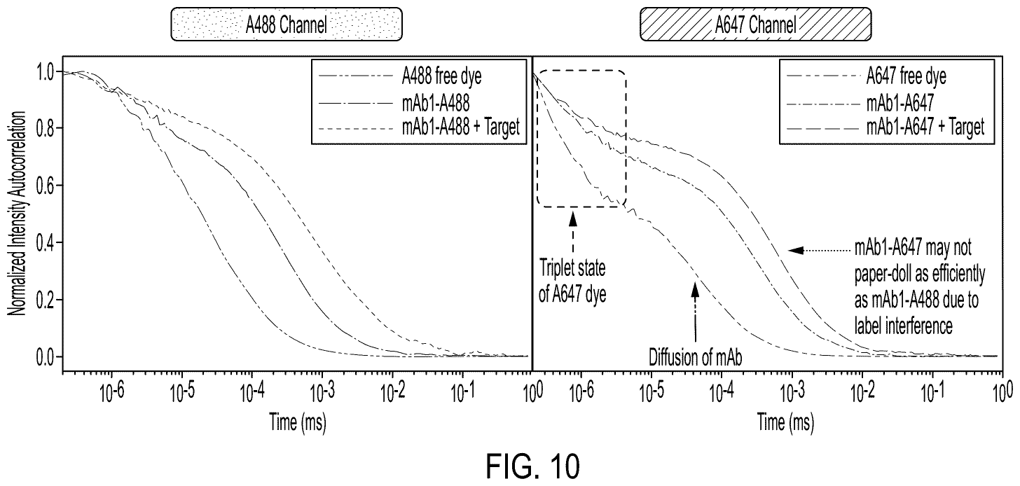

FIG. 10 shows the FCS auto-correlation curves of free dye, dye-conjugated-mAb1 and mAb1 complexes exhibited the expected trend of diffusion rates. mAb1-A488 (solid purple trace) and mAb1-A647 (solid red trace) exhibit slower diffusion than free Alexa Fluor 488 (dashed blue trace) and free Alexa Fluor 647 (dashed red trace). The FCS auto-correlation curves exhibit two transitions, the first of which represents the triplet state, an intrinsic property of the fluorophore, and the second of which is due to diffusion of the particle, the labeled mAb. The complexes between mAb1 and target exhibited the slowest decay, indicating large complex formation. The purple trace in the Alexa Fluor 647 channel shifts out a bit less than the red trace in the Alexa Fluor 488 channel, which may indicate that Alexa Fluor 647 interferes with complex formation in the mAb1-A647 sample.

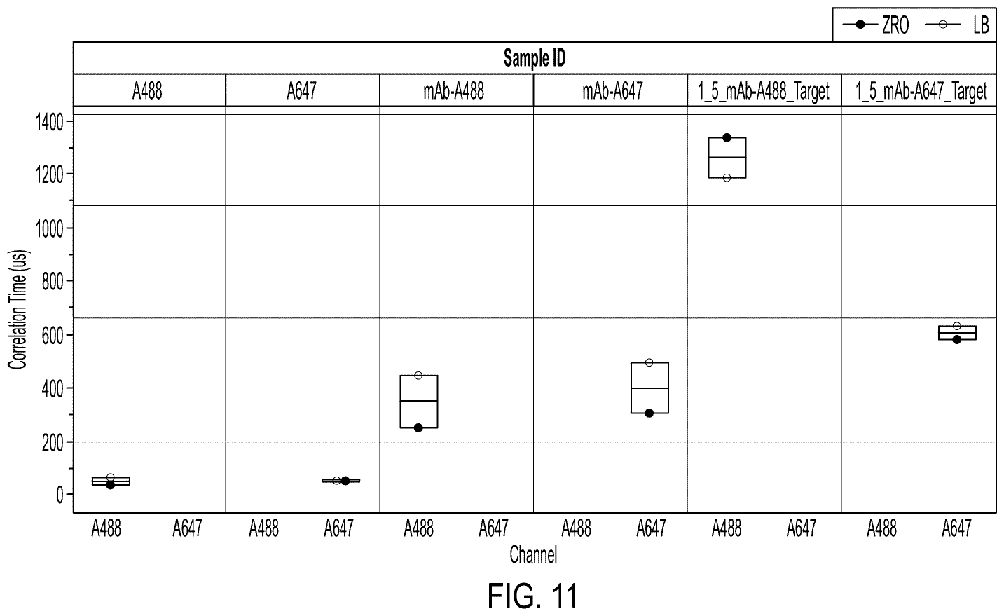

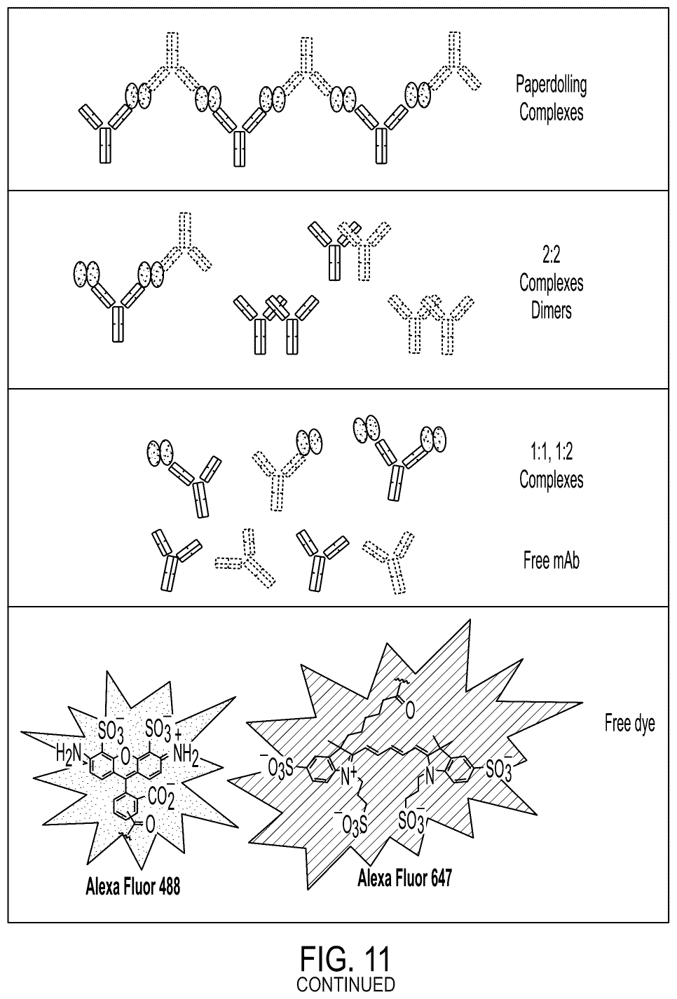

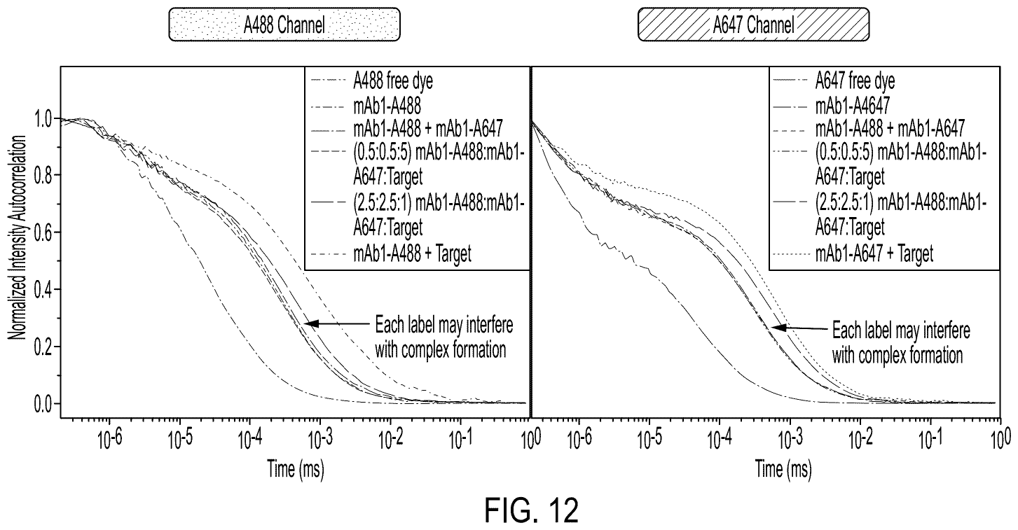

FIG. 11 shows that Alexa Fluor 488 and Alexa Fluor 647 controls diffuse rapidly and are only observed in their respective channels. Labeled antibodies exhibited longer correlation times of about 400 s, which is consistent with hydrodynamic radii of about 4 to about 6 nm. mAb1-A647 complexed with target exhibited correlation times that were slightly larger than mAb-only controls, which suggests that 1:1 or 1:2 complexes were formed. No species that represented mAb dimers or 2:2 complexes were observed. Alexa Fluor 488 and Alexa Fluor 647 controls were only observed in their respective channels. mAb1-A488 complexed with target exhibited the longest correlation times, which was consistent with larger complexes and, potentially, paper-dolling complexes. FIG. 12 shows that mAb1 complexes had higher complexes between mAb1 labeled with two different dyes formed smaller complexes than mAb1 complexed with itself using a single dye, which may indicate that each label interferes with complex formation.

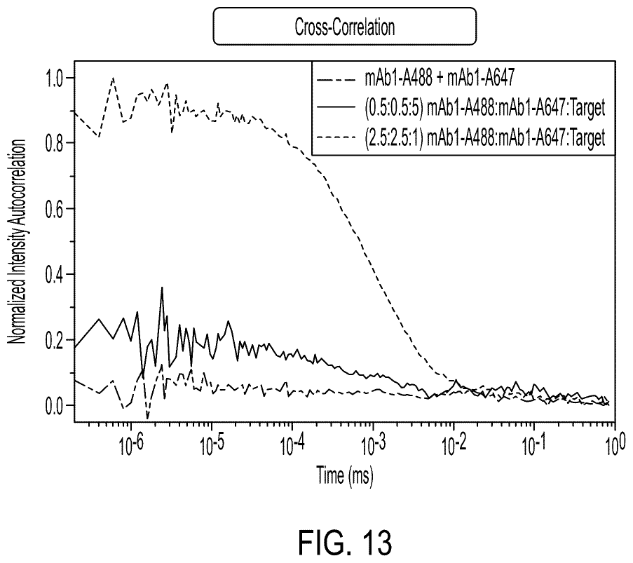

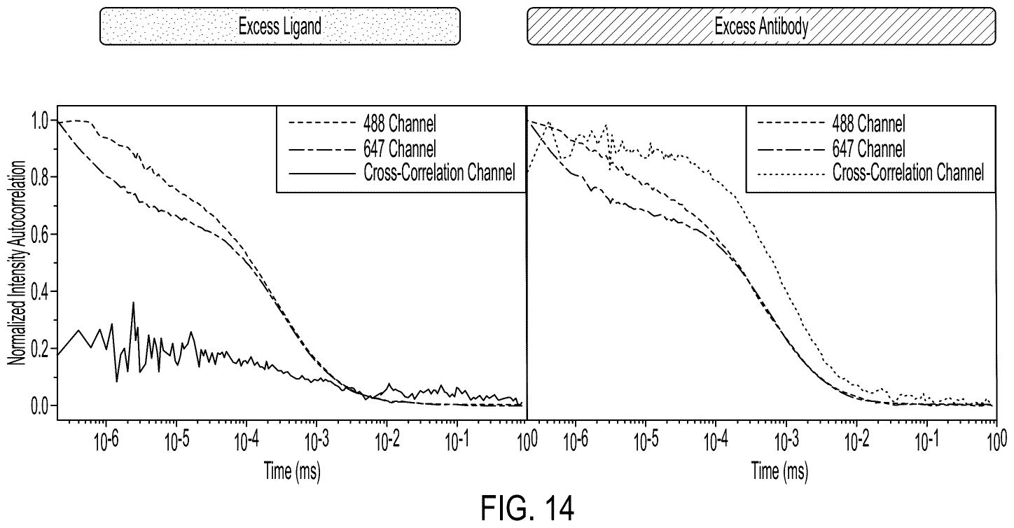

FIG. 13 shows that the mixed antibody (e.g., mAb1-A488 and mAb1-A647) cross-correlation negative control sample did not cross-correlate. FIG. 13, FIG. 14 and Table 1 indicate that the excess antibody sample (e.g., 2.5:2.5:1 mAb1-A488:mAb1-647:Target) exhibited the strongest cross-correlation.

TABLE 2

| | | Amp. | Cross-Correlation |
|---|---|---|---|
| | | Number | Diffusion Time |
| Sample | Description | Particles | (μs) |
| mAb1-A488 + | Cross-Correlation | 0.5 | — |
| mAb1-A647 | Negative Control | | |
| Excess Ligand | Low Paper-dolling | 0.3 | 880 |
| Excess mAb | Medium Paper-dolling | 2.4 | 810 |

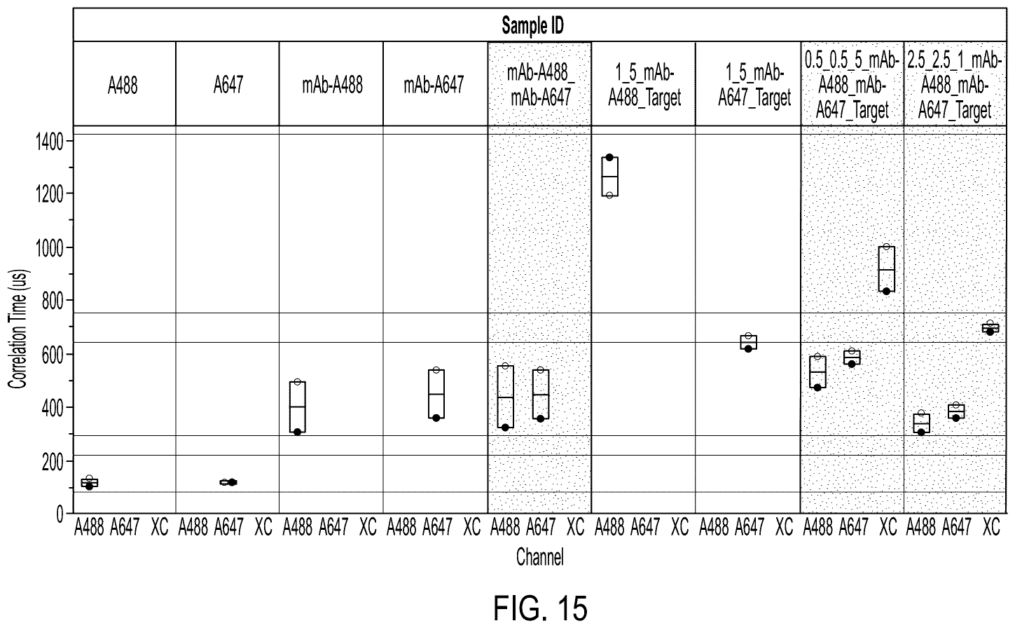

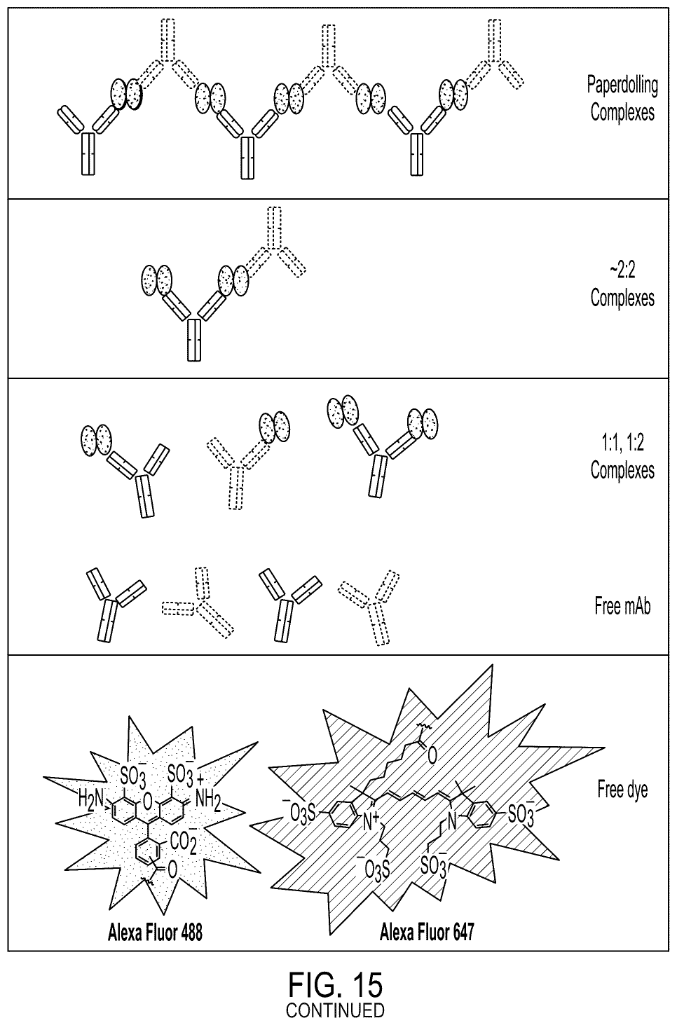

FIG. 15 shows that the cross-correlation (e.g., XC) times were consistently longer than the auto-correlation times, which indicated detection of co-localized (e.g., interacting) particles, because the cross-correlation times were blind to non-interacting species.

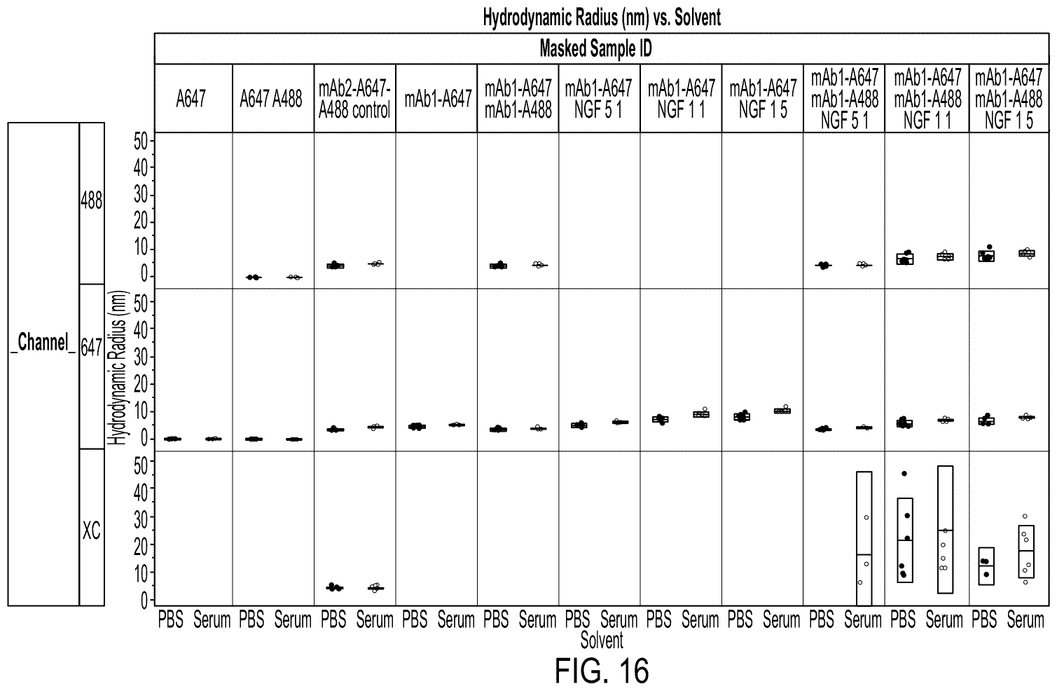

FIG. 16 shows FCS/FCCS data from mAb1:NGF samples. The data demonstrate that mAb1:NGF samples exhibit similar hydrodynamic radii in both PBS and serum across all channels.

What is claimed is:

1. A method of determining hydrodynamic radius in a sample, comprising:
a) contacting the sample with at least one unique fluorophore capable of binding to a protein;
b) measuring correlation times of the sample using a confocal microscope; and
c) determining the hydrodynamic radius in the sample based on the correlation times.

2. The method of claim 1, wherein said correlation times are determined using fluorescence correlation spectroscopy (FCS).

3. The method of claim 1, wherein the method is used to estimate protein-ligand stoichiometry in the sample.

4. The method of claim 2, wherein a fit model is used to determine said correlation times.

5. The method of claim 4, wherein said fit model is chosen from the group consisting of a triplet fit model, translation fit model or a combination thereof.

6. The method of claim 1, wherein said protein is an antibody.

7. The method of claim 1, wherein said protein is a monoclonal antibody.

8. The method of claim 1, wherein two unique fluorophores exhibit non-overlapping emission spectra.

9. The method of claim 1, wherein two unique fluorophores comprise Alexa Fluor 488, Alexa Fluor 647 or both.

10. The method of claim 1, wherein said correlation times are chosen from the group consisting of cross-correlation times, auto-correlation times or a combination thereof.

11. The method of claim 1, wherein said sample is serum.

12. The method of claim 1, wherein said sample comprises a biological system.

13. A method of determining hydrodynamic radius in a sample, comprising:
a) contacting the sample with at least one unique fluorophore capable of binding to a protein;
b) measuring cross-correlation and/or auto-correlation times of the fluorophores within the sample using a confocal microscope capable of fluorescence correlation spectroscopy (FCS); and
c) determining the hydrodynamic radius in the sample based on the correlation times.

14. The method of claim 13, wherein said cross-correlation and/or auto-correlation times are determined using fluorescence correlation spectroscopy (FCS).

15. The method of claim 13, wherein the method is used to estimate protein-ligand stoichiometry in the sample.

16. The method of claim 14, wherein a fit model is used to determine said cross-correlation and/or auto-correlation times.

17. The method of claim 16, wherein said fit model is chosen from the group consisting of a triplet fit model, translation fit model or a combination thereof.

18. The method of claim 13, wherein said protein is an antibody.

19. The method of claim 13, wherein said protein is a monoclonal antibody.

20. The method of claim 13, wherein two unique fluorophores exhibit non-overlapping emission spectra.

21. The method of claim 13, wherein two unique fluorophores comprise Alexa Fluor 488, Alexa Fluor 647 or both.

22. The method of claim 13, wherein said sample is serum.

23. The method of claim 13, wherein said sample comprises a biological system.

24. A method of estimating protein-ligand stoichiometry in a sample, comprising:
a) contacting the sample with at least one unique fluorophore capable of binding to a protein;
b) measuring cross-correlation and/or auto-correlation times of the fluorophores within the sample using a confocal microscope capable of fluorescence correlation spectroscopy (FCS); and
c) estimating the protein-ligand stoichiometry in the sample based on the correlation times.

25. The method of claim 24, wherein a fit model is used to determine said cross-correlation and/or auto-correlation times.

26. The method of claim 25, wherein said fit model is chosen from the group consisting of a triplet fit model, translation fit model or a combination thereof.

27. The method of claim 24, wherein said protein is an antibody.

28. The method of claim 24, wherein said protein is a monoclonal antibody.

29. The method of claim 24, wherein two unique fluorophores exhibit non-overlapping emission spectra.

30. The method of claim 24, wherein two unique fluorophores comprise Alexa Fluor 488, Alexa Fluor 647 or both.

31. The method of claim 22, wherein said sample is serum.

32. The method of claim 22, wherein said sample comprises a biological system.

33. A method of determining hydrodynamic radius in a sample, comprising:
a) labeling a protein with a first fluorophore;
b) labeling a ligand of the protein with a second fluorophore;
c) combining the labeled protein and the labeled ligand in said sample;
d) measuring correlation times of the sample using a confocal microscope capable of FCS; and
e) determining the hydrodynamic radius in the sample based on the correlation times.

34. The method of claim 33, wherein said correlation times are determined using fluorescence correlation spectroscopy (FCS).

35. The method of claim 33, wherein the method is used to estimate protein-ligand stoichiometry in the sample.

36. The method of claim 33, wherein a fit model is used to determine said correlation times.

37. The method of claim 36, wherein said fit model is chosen from the group consisting of a triplet fit model, translation fit model or a combination thereof.

38. The method of claim 33, wherein said protein is an antibody.

39. The method of claim 33, wherein said protein is a monoclonal antibody.

40. The method of claim 33, wherein said first and second fluorophores exhibit non-overlapping emission spectra.

41. The method of claim 33, wherein said first and second fluorophores comprise Alexa Fluor 488, Alexa Fluor 647 or both.

42. The method of claim 33, wherein said correlation times are chosen from the group consisting of cross-correlation times, auto-correlation times or a combination thereof.

43. The method of claim 33, wherein said sample is serum.

44. The method of claim 33, wherein said sample comprises a biological system.

45. A method of estimating protein-ligand stoichiometry in a sample, comprising:
a) labeling a protein with a first fluorophore;
b) labeling a ligand of the protein with a second fluorophore;
c) combining the labeled protein and the labeled ligand in the sample;
d) measuring cross-correlation and/or auto-correlation times of the sample using a confocal microscope capable of FCS; and
e) estimating the protein-ligand stoichiometry in the sample based on the correlation times.

46. The method of claim 45, wherein said cross-correlation and auto-correlation times are determined using fluorescence correlation spectroscopy (FCS).

47. The method of claim 45, wherein a fit model is used to determine said cross-correlation and/or auto-correlation times.

48. The method of claim 47, wherein said fit model is chosen from the group consisting of a triplet fit model, translation fit model or a combination thereof.

49. The method of claim 45, wherein said protein is an antibody.

50. The method of claim 45, wherein said protein is a monoclonal antibody.

51. The method of claim 45, wherein said first and second fluorophores exhibit non-overlapping emission spectra.

52. The method of claim 45, wherein said first and second fluorophores comprise Alexa Fluor 488, Alexa Fluor 647 or both.

53. The method of claim 45, wherein said sample is serum.

54. The method of claim 45, wherein said sample comprises a biological system.

55. A method of determining hydrodynamic radius in a sample, comprising:
a) labeling a protein with a first fluorophore;
b) labeling a secondary labeled reporter with a second fluorophore;
c) combining the labeled protein and secondary labeled reporter in said sample;
d) measuring cross-correlation and/or auto-correlation times of the sample using a confocal microscope capable of fluorescence correlation spectroscopy (FCS); and
e) determining the hydrodynamic radius in the sample based on the correlation times.

56. The method of claim 55, wherein the method is used to estimate protein-ligand stoichiometry in the sample.

57. The method of claim 55, wherein a fit model is used to determine said cross-correlation and/or auto-correlation times.

58. The method of claim 57, wherein said fit model is chosen from the group consisting of a triplet fit model, translation fit model or a combination thereof.

59. The method of claim 57, wherein said protein is an antibody.

60. The method of claim 57, wherein said protein is a monoclonal antibody.

61. The method of claim 57, wherein said first and second fluorophores exhibit non-overlapping emission spectra.

62. The method of claim 57, wherein said first and second fluorophores comprise Alexa Fluor 488, Alexa Fluor 647 or both.

63. The method of claim 57, wherein said sample is serum.

64. The method of claim 57, wherein said sample comprises a biological system.
